(19) United States
(12) Patent Application Publication
(10) Pub. No.: US 2012/0116370 A1
(43) Pub. Date: May 10, 2012

(54) Surgical Instrument

(75) Inventor: Peter Tschida, Barenthal (DE)

(73) Assignee: TMT TSCHIDA MEDIZIN TECHNIK, Barenthal (DE)

(21) Appl. No.: 13/384,323

(22) PCT Filed: Jul. 2, 2010
(86) PCT No.: PCT/EP2010/004009

§ 371 Date: Jan. 18, 2012

(30) Foreign Application Priority Data
Jul. 29, 2009 (EP) 09009773.4

(51) Int. Cl. A61B 17/00

(52) U.S. Cl. 606/1

(57) ABSTRACT

The invention relates to a surgical instrument, for example, surgical scissors, having a casing and a core that is slidable in the casing. The invention proposes arranging a flushing connection at the distal end, in order to be able to clean the casing completely for the entire length thereof, starting at the distal end.

### FIELD OF THE INVENTION

The invention relates to a surgical instrument.

### BACKGROUND OF THE INVENTION

The laying open document DE 42 28 619 A1 discloses a surgical instrument having a casing and a core that is arranged in the casing and is moveable with respect to the casing for actuating the instrument. In this instance, core means an element that is moveable within the casing and that transmits a pulling and/or compressing force. The casing of the known surgical instrument is a tube,and the core a rod. At a proximal end, the known surgical instrument has jaws with two jaw legs that are pivotable one against the other and that can be pivoted by displacing the core against the casing in order to open or close the jaws. The jaws can be forceps or scissors, for example. Both jaw legs can be pivotable or one jaw leg can be fixed and the other jaw leg can pivot. At one distal end, the known surgical instrument has a forceps handle by means of which the core is displaced against the casing and the jaws close and open, that is to say the instrument can be operated.

The known surgical instrument is, like all surgical instruments, a high precision tool with moveable parts that are arranged in the smallest of spaces at the end of the casing and within the casing and are therefore difficult to clean. However, thorough cleaning is indispensable for surgical instruments. Capillary forces draw contaminants such as blood, secretions, tissue fragments, etc. into an intermediate space between the core and the casing. The high pressure that is sometimes prevalent at the surgical site during some surgeries increases the penetration of contaminants into the intermediate space between the core and the casing of the surgical instrument. For purposes of cleaning, the casing and the core can be simply separated from one another in the known surgical instrument. The casing is fastened to the forceps handle with a connecting nut that suspends the rod that forms the core and is protected by the casing as long as the casing is attached to the forceps handle.

### SUMMARY OF THE INVENTION

The problem addressed by the invention is that of suggesting a surgical instrument of the generic type in question that can be cleaned internally without taking it apart, “internally” meaning the interior of the casing.

The surgical instrument according to the invention has a flush connection on the distal end of the casing, it also being possible for said flush connection to be arranged on the core, in such a manner that a flushing solution enters into the casing through the flush connection on the distal end. An advantage of the invention is that the surgical instrument does not have to be taken apart for cleaning, but rather a flushing line need only be connected to the flush connection. An expert does not have to carry out this procedure since the instrument can be flushed and cleaned by unschooled personnel. A further advantage is that during cleaning, there is no risk in damaging the instrument or of reassembling it incorrectly owing to the fact that the instrument does not have to be taken apart for cleaning. The interior of the casing is flushed beginning at the distal end over the entirety of the casing, the invention dispensing with a dead space in the distal end of the casing through which flushing solution, which is conducted through the flush connection, does not necessarily flow. The meaning of dead space is a region of the casing that is contaminated and that can also be reached by the flushing liquid, yet in which a current is, however, not necessarily formed because the flushing fluid can enter into the dead space from only one side from which it must also exit again. The invention thus has the advantage that the interior of the casing can be cleaned simply, thoroughly, and reliably by unschooled personnel, and furthermore that contaminations can be completely flushed out.

A scissors handle or a forceps handle is provided for operation of the surgical instrument, this listing not being exhaustive. A handle of a pair of scissors has arms that have finger holes, in particular, that preferably can be gripped by fingers at their ends. A forceps handle is gripped with the hand. In extending the casing, the handles can also be arranged in parallel or at an angle (offset) to one another or at an angle to the casing. A forceps handle can also be directed proximally instead of distally from the distal end of the casing. Other configurations are conceivable. Two moveable handles or the like or one moveable handle or the like can be provided, which moveable handle is moveable against a fixed handle or the like.

In a preferred embodiment of the invention, the flush connection is accessible without the instrument having been taken apart, that is to say without the scissors or forceps handle or another operating mechanism or operating device having been removed, which is not, however, obligatory for the invention. Essential for the invention is the flush connection that ensures a flushing of the interior of the casing over the entire length beginning at the distal end and without dead space. The flush connection has, in particular, a luer connection as a plug connection or preferably as a secure luer lock.

The casing of the surgical instrument according to the invention can be rigid, as in a tube, or flexible, just as the core can be that can consist of a rod, wire, or possibly even wire rope or can moreover be composed of plastic. The casing and core are particularly rigid in the longitudinal direction in which the force of the operation of the instrument must be transmitted in order to transfer a shifting of the core with respect to the casing begun at the distal end to the proximal end with as little loss as possible.

One embodiment of the invention provides for a hollow pipe that is longitudinally connected to the core in a fixed manner and that has the flush connection arranged on its distal end. The hollow pipe has a lateral outlet opening for flushing liquid and it opens into the core of the surgical instrument. Flushing liquid introduced via the flush connection flows through the hollow pipe and flows out through its lateral outlet opening into the casing through which the flushing solution flows around the core to the proximal end of the casing, where it then flows out. The hollow pipe can be rigid, that is to say it can be a tube, or it can be flexible, although longitudinally rigid. The hollow pipe serves to conduct the flushing solution through the flush connection into the casing of the surgical instrument and furthermore to transfer force to the core for the operation of the surgical instrument. The hollow pipe can also be regarded as part of the core.

In a preferred embodiment of the invention, if the surgical instrument is inoperative, the outlet opening of the hollow pipe is arranged close to a proximal end of a distal end piece of the casing, that is to say on the distal end of the interior space of the casing, which distal end can have distance from the distal end of the casing. This arrangement of the outlet opening ensures that the flushing liquid enters into the casing at the distal end of the interior space of the casing and further establishes a full flushing beginning at the distal interior end of the casing.

A further development of the invention provides for a plurality of longitudinally-distributed outlet openings of the hollow pipe, the outlet openings also being preferably arranged in the peripheral direction of the hollow pipe. The outlet openings can be distributed uniformly or non-uniformly across the hollow pipe, the hollow pipe being a perforated sleeve. A plurality of outlet openings enlarges the entry cross-section for the flushing liquid from the hollow pipe into the casing and ensures an exiting of the flushing liquid from the hollow pipe into the casing at its internal, distal end upon different displacement positions of the core in the casing.

An embodiment of the invention provides for a narrow gap between the casing and the core or, generally, a small, free cross-section between casing and core. A high rate of flow upon flushing and an accordingly favorable cleaning effect are attained in this manner.

For sound cleaning of the exterior side of the surgical instrument, a further embodiment of the invention provides a handle, the surface of which has holes that are arranged so as to be uniformly or non-uniformly distributed across the surface thereof, which holes run from the outside to the inside of the instrument. In this instance, “surface” means an outer surface or handle surface of the handle. The holes in the handle make entry of cleaning solution possible that is sprayed exteriorly onto the instrument in such a manner that the cleaning solution reaches an actuating mechanism by traveling through the handle, which actuating mechanism is positioned in an intermediate space between the handle and the instrument, and further said cleaning solution also reaches the exterior side of the surgical instrument. The holes in the handle reduce a barrier to the surgical instrument and to any actuating mechanism against externally sprayed cleaning solution through the handle. The handle serves for holding and/or operating the surgical instrument and it can be fixed or moveable such as a scissors handle or a forceps handle. This embodiment of the invention improves the overall cleaning of the exterior and interior of the surgical instrument. Said handle in a surgical instrument of the generic type in question can, however, also be provided on the distal end of the casing independent of the flush connection.

Another embodiment of the invention provides a seal at the distal end between the casing and the core on a circumference smaller than the interior circumference of the casing. The circumference is the diameter when the cross-section is circular. To this end, the core can have a smaller circumference at the distal end or the hollow pipe for guiding the flushing solution at the distal end of the core can have a circumference that is smaller than that of the core. Owing to the sealing on a smaller circumference, there is diminished friction and accordingly reduced actuation force is required. This embodiment of the invention can also be provided on the distal end of the casing of a surgical instrument of the generic type in question independent of the flush connection.

A further embodiment of the invention provides for an overload safety device. An overload safety device serves to limit force on the casing and the core if their movement against one another is blocked prior to reaching an end position when, for example, a jaw cannot be closed on the proximal end of the surgical instrument. As an overload safety device, one embodiment of the invention provides an elastic element in a flow of force for or upon actuation of the instrument, that is to say an elasticity in the transmission of force of a scissors or forceps handle or another actuating mechanism or actuating device to the casing or the core. If the core on the proximal end is blocked from displacement with respect to the casing, the increase of force working against the casing and the core is less upon a further closing of the scissors or forceps handle or another actuating mechanism or actuating device than without an elastic element or without an overload safety device. The elastic element can be a silicone sleeve, another elastic synthetic material or rubber sleeve, a spring, in particular a helical spring, a disk spring or a laminated disk spring. This list is not exhaustive.

A further embodiment of the invention provides that the elastic element encloses the flush connection and acts against a support of the flush connection, that is to say, for example, abuts against an annular bead seat or a flange of the flush connection. The overload safety device can thereby be housed in a space-saving manner, it only minimally lengthening the surgical instrument and not enlarging its diameter at all.

### BRIEF DESCRIPTION OF THE DRAWINGS

The invention is described in greater detail in the following using the one embodiment represented in the drawing, which shows in

FIG. 1 a surgical instrument according to the invention shown in axial view, and in

FIG. 2 the surgical instrument in the view of a representation that has been rotated 90° about the longitudinal axis of said surgical instrument.

### DETAILED DESCRIPTION OF THE PREFERRED EMBODIMENTS

A pair of surgical scissors has been selected as an exemplary embodiment of a surgical instrument 1 according to the invention, other surgical instruments such as a surgical forceps (not shown) being possible. The surgical instrument 1 has a tube 2 as a casing 3 and a wire rod 4 as a core 5 that is moveable within the casing 3. At a distal end, the core 5 is rigidly, for example by means of welding, connected to a perforated sleeve 6, in other words a tube 2 that has a plurality of holes, that can also be interpreted as a hollow pipe 7. Holes are distributed over the circumference and longitudinally over the hollow pipe 7 and form outlet openings 8. A distal section of the hollow pipe 7 has no holes. The hollow pipe 7 has a diameter that is smaller than that of the core 5 and coaxially extends the core 5 at the distal end.

A distal end of the hollow pipe 7 protrudes from a distal end of the casing 3 and it is connected to a flush connection 9 on the distal end of the hollow pipe 7 that protrudes from the casing 3. In the exemplary embodiment, the flush connection 9 is a luer lock, which is not, however, obligatory for the invention. Flushing liquid, which is conducted through the flush connection 9, flows through the hollow pipe 7 and exits by flowing through the outlet openings 8 of said hollow pipe into the casing 3. From there, the flushing liquid flows between the core 5 and the casing 3 to the proximal end from whence it then exits. Since the core 5 largely fills the casing 3, only a small gap or free cross-section remains through which the flushing liquid must flow. The flushing liquid therefore flows with a greater rate of speed and has a very favorable cleaning effect.

A distal end piece 10 is fixed, for example by means of welding; to the casing 3 at the distal end of said casing 3. The end piece 10 is cylindrical and has the same exterior diameter as the casing 3. It is provided with a cylindrical, axial clearance hole through which the hollow pipe 7 is axially moveable and passes in a sealed manner. The outlet openings 8 of the hollow pipe 7 extend up into the distal end piece 10 of the casing 3 in such a manner that outlet openings 8 of the hollow pipe are always positioned at a distal, interior end of the casing 3, even if the hollow pipe 7 is displaced with the core 5 into the casing 3. This ensures an exit for the flushing liquid from the hollow pipe 7 into the casing 3 at the distal, interior end of the casing 3, thereby safeguarding that contaminants such as blood, secretions, and tissue fragments are reliably rinsed out on the entire length of the casing 3, beginning directly at the internal, distal end thereof. A thorough and complete cleaning of the interior space of the casing 3 of the surgical instrument 1 is thereby guaranteed. The distal, interior end of the casing 3 is located at the proximal end of the distal end piece 10.

To create a seal between the distal end piece 10 of the casing 3 and the hollow pipe 7, a sealing ring, which is not shown, can be provided in a circumferential groove in the axial clearance hole of the distal end piece 10. The seal occurs at the hole-free distal section of the hollow pipe 7 that extends into the distal end piece 10. The seal of the hollow pipe 7 in the distal end piece 10 is achieved on the diameter of the hollow pipe 7, that is to say on a diameter that is smaller than the diameter of the core 5 and the interior diameter of the casing 3. The seal on a smaller diameter reduces friction upon moving the hollow pipe 7 in the axial direction.

For purposes of actuation, that is to say to move the core 5 in or against the casing 3, the surgical instrument 1 has a forceps handle 11. The forceps handle 11 has two hand grips 12 that are shaped like cylindrical shells and that are arranged opposite one another on both sides of the casing 3. The hand grips 12 are arranged longitudinally to the core 3 and at an acute angle in the direction of the proximal end pointing diagonally outward. Distal ends of the hand grips 12 are attached at the proximal ends by means of spring elements 13 that are configured similarly to disk springs. For the formation of the spring elements 13, a tube 27 composed of an elastic material is slit on two sides, which are opposite one another, in the longitudinal direction over a majority of its length, the slits being open on one end. Half cylinder shells are formed by the slits, which half cylinder shells are somewhat curved away from one another in such a manner that they extend diagonally outward in the proximal direction. The half cylinder shells are laterally pared over a portion of their length, that is to say they are narrowed. The half cylinder shells form the spring elements 13 that hold and guide the hand grips 12. A slit-free, distal section 28 of the tube 27 that is closed in the circumferential direction encases the distal end of the casing 3 and is attached on the core 3.

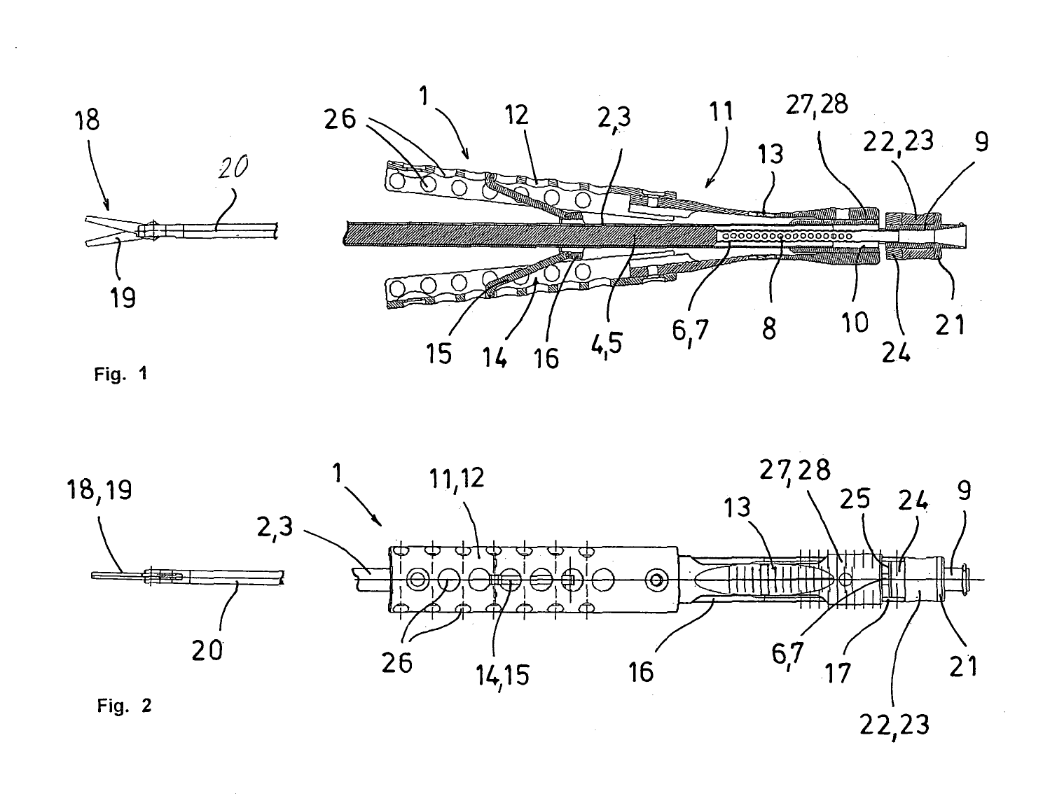

The forceps handle 11 has a lever mechanism 14 that changes a movement of the hand grip 12, which movement is transverse to the casing 3 and occurs upon pressing together the forceps handle 11 in a longitudinal direction, into the movement of the core 5 within or against the casing 3. For each hand grip 12, the lever mechanism 14 has a lever 15 that is articulated on the hand grip 12. The levers 15 extend obliquely from the hand grips 12 in the distal direction inward to the casing 3 where they are articulated on a proximal end of a sliding sleeve 16. The sliding sleeve 16 is laterally cut over a large portion of its length, which is why it appears interrupted in FIG. 1. A distal end 17 of the sliding sleeve 16 transmits the sliding movement onto the flush connection 9 and via that and the hollow pipe 7 to the core 3.

The actuation of the surgical instrument 1 with the forceps handle 11 is not necessary since there are other actuating mechanisms or general actuating devices that are also possible for moving the core 5 into or against the casing 3. For example, a moveable hand grip can be moved against a fixed hand grip, the hand grips can be arranged in a manner opposite that which is shown with the joints on the proximal ends of the hand grips and the hand grips being arranged distally protruding over the distal end of the casing 3 (not shown). The forceps handle 11 can also be arranged transverse to or at an angle to the casing 3 or the surgical instrument 1 has a scissors handle (not shown). Rather than being exhaustive, this list addresses only a few possibilities of an actuating device or actuating mechanism for the surgical instrument 1.

In the exemplary embodiment, the surgical instrument 1 has a scissors at its proximal end, which is why the surgical instrument 1 can also be characterized as a pair of surgical scissors. The scissors 18 have two blades 19 that are mounted pivotably in a proximal, forked end piece 20 of the casing 3. The blades 19 are articulated on the core 5 and are pivoted by moving the core 5 within or against the casing 3. A pair of scissors 18 with a fixed and a pivotable blade (not shown) is also conceivable. Instead of scissors, the surgical instrument 1 can, for example, also have a forceps with two jaws instead of the blades 19 (not shown). This list is also not exhaustive.

The flush connection 9 has a flange that forms a support 21. An elastic element 22 is arranged on the flush connection 9 on a proximal side of the support 21, the elastic element 22 being a silicon sleeve or another elastic sleeve composed of plastic or rubber in the exemplary embodiment. The distal end 17 of the sliding sleeve 16 abuts the proximal end of the elastic element 22, the distal end 17 of the sliding sleeve 16 or the entirety of the sliding sleeve 16 is moveable on the flush connection 9. Upon pressing the hand grips 12 of the forceps handle 11, the lever 15 displaces the sliding sleeve 16 distally against the casing 3. The distal displacement of the sliding sleeve 16 is transmitted to the support 21 of the flush connection 9 by means of laying its distal end 17 on the elastic element 22. The flush connection 9 distally pulls the hollow pipe 7, thereby likewise distally moving the core 5 into the casing 3. The pair of scissors is closed in this manner. Should the pair of scissors not (fully) close owing to resistance, the elastic element 22 provides resilience when the hand grips are squeezed together and said elastic element is elastically compressed. The resilience of the elastic element 22 limits an increase in pressure, thereby preventing damage to the scissors 18 and to the surgical instrument 1 overall in the event that the scissors 18 cannot be (fully) closed. The elastic element 22 can therefore be regarded as an overload safety device 23.

A ring 24 is rigidly attached in the distal end 17 of the sliding sleeve 16, which ring is moveable on the flush connection 9. The flush connection 9 has at its proximal end a flange that serves as a stop unit 25 against which the lock ring 24 abuts. Upon an opening of the scissors, a movement in the proximal direction from the sliding sleeve 16 to the flush connection 9 and from there to the hollow pipe 7 and the core 5 and the converse is transferred by means of the lock ring 24 and the stop unit 25. The surgical instrument 1 is opened by the spring force of the spring elements 13.

The hand grips 12 of the forceps handle 11 outwardly shield the casing 3 as if they were shields. The hand grips 12 are provided with holes 26 that are arranged distributed over the surface of the hand grips 12. Cleaning fluid sprayed onto the surgical instrument 1 can enter the holes 26 of the hand grips 12 from the outside and reach the lever mechanism 14 that is arranged on the interior side of the hand grips 12. The holes 26 of the hand grips 12 make it possible to clean or improve cleaning the core 3 and the lever mechanism 14 from the exterior. The cleaning fluid for external spraying can be the same liquid used as the flushing solution for flushing the interior of the casing 3. Different terms for the liquid are used owing to its type of use and the manner in which it is introduced and not necessarily because different solutions are concerned.

1-11. (canceled)

12. A surgical instrument with a casing and a core that is arranged within the casing that is movable with regard to the casing, wherein the casing has a flush connector at a distal end.

13. The surgical instrument of claim 12, wherein the flush connector is accessible when the instrument has not been taken apart.

14. The surgical instrument of claim 12, wherein the flush connector is arranged on a distal end of a hollow pipe that is axially connected in a fixed manner to the core and that has a lateral outlet opening for flushing solution that flows into the casing.

15. The surgical instrument of claim 14, such that when the instrument is not being actuated, the outlet opening is located close to a distal, interior end of the casing.

16. The surgical instrument of claim 14, wherein the hollow pipe has a plurality of longitudinally distributed outlet openings.

17. The surgical instrument of claim 12, defining a gap between the casing and the core, wherein the size of the gap is small in relation to a cross-section.

18. The surgical instrument of claim 12, wherein the surgical instrument has a hand grip for actuation that has holes arranged distributed over its surface, which holes pass from outside to inside in relation to the instrument.

19. The surgical instrument of claim 12, wherein the instrument has at the distal end a seal of the core in the casing which seal seals with a circumference that is less than that of the interior circumference of the casing.

20. The surgical instrument of claim 12, wherein the surgical instrument has an overload safety device.

21. The surgical instrument of claim 20, wherein the overload safety device has an elastic element in a flow of force for actuating the instrument.

22. The surgical instrument of claim 21, wherein the elastic element encloses the flush connection and acts against a support of the flush connection.

23. A surgical instrument adapted to be cleaned without having to taken apart which comprises:
a casing;
a core that is disposed within the casing and that is slidable within the casing wherein a gap between the casing and core defines a fluid path along the length of the casing; and
a flush connector that is attached at a distal end of the instrument and that is in fluid communications with the gap and wherein the flush connector is accessible when the instrument has not been taken apart.

24. The surgical instrument of claim 23, wherein the flush connector is arranged on a distal end of a hollow pipe that is axially connected in a fixed manner to the core and that has a lateral outlet opening for flushing solution that flows into the casing.

25. The surgical instrument of claim 24, such that when the instrument is not being actuated, the outlet opening is located close to a distal, interior end of the casing.

26. The surgical instrument of claim 23, wherein the surgical instrument has a hand grip for actuation that has holes arranged distributed over its surface, which holes pass from outside to inside in relation to the instrument.

27. The surgical instrument of claim 23, wherein the instrument has at the distal end a seal of the core in the casing which seal seals with a circumference that is less than that of the interior circumference of the casing.

28. A method of cleaning a surgical instrument that includes a casing and a core that is disposed within the casing and that is slidable within the casing wherein a gap between the casing and core defines a fluid path along the length of the casing, the method comprising the steps of:
attaching a flush connector to a distal end of the instrument;
introducing a flushing solution through the flush connector and into an interior of the casing and over the entire casing wherein the flush connector is accessible when the instrument has not been taken apart.

29. The method of claim 28, wherein the surgical instrument has a hand grip for actuation that has holes arranged distributed over its surface, which holes pass from outside to inside in relation to the instrument, and the method further comprises the step of spraying cleaning solution exteriorly onto the instrument whereby the cleaning solution reaches an actuating mechanism, which is located between the handle and the instrument, by traveling through the holes in the handle.

30. The method of claim 28, wherein the flush connector is arranged on a distal end of a hollow pipe that is axially connected in a fixed manner to the core and that has a lateral outlet opening for the flushing solution that flows into the casing.

31. The method claim 30, such that when the instrument is not being actuated, the outlet opening is located close to a distal, interior end of the casing.
